(19) United States
(12) Patent Application Publication
(10) Pub. No.: US 2011/0111112 A1
(43) Pub. Date: May 12, 2011

(54) OIL-AND-FAT COMPOSITION, AND FOOD OR BEVERAGE CONTAINING THE OIL-AND-FAT COMPOSITION

(75) Inventors: Ryuuji Noda, Kanagawa (JP); Seiji Sekine, Kanagawa (JP); Chika Ogasawara, Kanagawa (JP); Hiroyuki Takeuchi, Kanagawa (JP); Junichi Ikuina, Kanagawa (JP); Takashi Toyoshima, Kanagawa (JP)

(73) Assignee: THE NISSHIN OILLIO GROUP, LTD., Tokyo (JP)

(21) Appl. No.: 12/448,622

(22) PCT Filed: Dec. 21, 2007
(86) PCT No.: PCT/JP2007/074707

§ 371 Date: Aug. 13, 2009

(30) Foreign Application Priority Data
Dec. 28, 2006 (JP) 2006-356388

(51) Int. Cl. A23D 9/00; A23L 2/52; A23L 1/30

(52) U.S. Cl. 426/590; 426/607

(57) ABSTRACT

Disclosed is an oil-and-fat composition having a high α-linolenic acid content, a hypertension-ameliorating effect, a good flavor, and good storage stability. Also disclosed is a food or beverage containing the oil-and-fat composition. The oil-and-fat composition comprises at least one plant oil A selected from flax oil, perilla oil and perilla seed oil, rice oil, and at least one plant oil B selected from rape seed oil, soybean oil and corn oil at a ratio among the plant oil A, rice oil and the plant oil B [i.e., a (plant oil A)/(rice oil)/(plant oil B) ratio] of 20-35/15-25/40-65 by mass, wherein the α-linolenic acid content in the constituent fatty acids contained in the oil-and-fat composition is 15 to 30 mass %.

### FIELD OF THE INVENTION

This invention relates to an oil-and-fat composition having a high α-linolenic acid content and a food or beverage containing the oil-and-fat composition.

### BACKGROUND ART

α-linolenic acid is a kind of n-3 polyunsaturated fatty acid and is called as an essential fatty acid, but it can not be synthesized in vivo, so that it should be consumed in foods.

Some of the vegetable oils have the α-linolenic acid content of not less than 50 mass % in the constituent fatty acids, and as the typical examples, flax oil (flaxseed oil), perilla oil (egoma oil) and perilla seed oil (shiso seed oil) can be given.

However, the oils having the α-linolenic acid content of not less than 50 mass % such as flax oil, perilla oil and perilla seed oil are easily deteriorated and have the problems of flavor and storage stability, therefore, it was difficult to realize the product commercialization. Further, in a manufacturing process of the vegetable oil, normally, a deodorization process is carried out, however, α-linolenic acid has a structure that tree double bonds are included, and due to this, it has also a problem that it is easily turned into trans fatty acid.

As a countermeasure for solving the above-mentioned problem, an edible oil is disclosed, which has the α-linolenic acid content of not less than 10 mass % obtained by adding a high-oleic oil-and-fat having the oleic acid content of not less than 60 mass % to flax oil having the α-linolenic acid content of not less than 50 mass % and the trans fatty acid content of not more than 4 mass %, and the ratio by mass of the oleic acid to the α-linolenic acid is controlled so as to be 1.0:0.2-0.6. (refer to Patent Literature 1)

Patent Literature 1 discloses that flax oil can be manufactured, which has a good flavor and the trans fatty acid content suppressed to not more than 4 mass %, if the deodorization process in the manufacturing process of flax oil is carried out in the condition that the deodorization temperature is 220 to 240 degrees C., the deodorization time is 60 to 90 minutes and the blowing amount of steam to the oil is 3.5 to 5.5 mass %.

Further, an oil-and-fat composition containing α-linolenic acid is disclosed, which includes rice oil having an odor masking effect (refer to Patent Literatures 2, 3).

Patent Literature 2 discloses an oil-and-fat composition comprising 30 to 60 weight % of an edible oil-and-fat derived from rice and 5 to 10 weight % of an oil-and-fat having α-linolenic acid content of not less than 45 weight %, and Literature 3 discloses an oil-and-fat composition comprising not more than 10 weight % of an oil-and-fat having α-linolenic acid content of not less than 50 weight %, and more than 60 weight % and not more than 85 weight % of an edible oil-and-fat derived from rice, of the whole oil-and-fat composition.

On the other hand, it is reported that α-linolenic acid plenteously contained in flax oil can decrease a risk of development of hypertension in human epidemiological study (refer to Non Patent Literature 1). Further, it is known that an edible oil-and-fat composition having a high α-linolenic acid content has a preventive effect against the hypertension (refer to Patent Literature 4).
Patent Literature 1: JP-A-2000-157170
Patent Literature 2: JP-A-2003-79314
Patent Literature 3: JP-A-2004-254588
Patent Literature 4: JP-A- S63 (1988)-36744
Non Patent Literature 1: Luc Djousse et al. “Dietary Linolenic Acid Is Associated With a Lower Prevalence of Hypertension in the NHLBI Family Heart Study.” Hypertension, volume 45, p. 368 to 373, 2005

### DISCLOSURE OF THE INVENTION

### Problems to be Solved by the Invention

However, as previously mentioned, the oils having the α-linolenic acid content of not less than 50% such as flax oil, perilla oil and perilla seed oil have the problems of flavor and storage stability, therefore, if the oil-and-fat composition is configured to have a high α-linolenic acid content so as to achieve the preventive effect against the hypertension, it becomes difficult to obtain the oil-and-fat composition having a good flavor, and good storage stability, even if the edible oils described in Patent Literatures 1 to 3 are used.

Therefore, it is an object of the invention to provide an oil-and-fat composition having a high α-linolenic acid content, a hypertension-ameliorating effect, a good flavor, and good storage stability, and a food or beverage containing the oil-and-fat composition.

### Means for Solving the Problems

In order to achieve the above-mentioned object, the invention provides an oil-and-fat composition, comprising: at least one vegetable oil A selected from flax oil, perilla oil and perilla seed oil; rice oil; and at least one vegetable oil B selected from rape seed oil, soybean oil and corn oil, at a ratio among the vegetable oil A, the rice oil and the vegetable oil B of 20-35/15-25/40-65 by mass, wherein; the α-linolenic acid content in the constituent fatty acids contained in the oil-and-fat composition is 15 to 30 mass %.

Further, in order to achieve the above-mentioned object, the invention provides an oil-and-fat composition, comprising: 20-35 mass % of at least one vegetable oil A selected from flax oil, perilla oil and perilla seed oil; and 15-25 mass % of rice oil, wherein; the α-linolenic acid content in the constituent fatty acids contained in the oil-and-fat composition is 15 to 30 mass %.

Further, in order to achieve the above-mentioned object, the invention provides an oil-and-fat composition, comprising: 30-53 mass % of at least one vegetable oil A selected from flax oil, perilla oil and perilla seed oil; and 70-47 mass % of rice oil, wherein; the α-linolenic acid content in the constituent fatty acids contained in the oil-and-fat composition is 15 to 30 mass %.

Further, in order to achieve the above-mentioned object, the invention provides a food or beverage containing the oil-and-fat composition according to the inventions.

### Advantages of the Invention

According to the invention, an oil-and-fat composition having a high α-linolenic acid content, a hypertension-ameliorating effect, a good flavor, and good storage stability, and a food or beverage containing the oil-and-fat composition can be provided.

### BEST MODE FOR CARRYING OUT THE INVENTION

### Oil-and-Fat Composition According to a First Embodiment of the Invention

An oil-and-fat composition according to a first embodiment of the Invention comprises at least one vegetable oil A selected from flax oil, perilla oil and perilla seed oil; rice oil; and at least one vegetable oil B selected from rape seed oil, soybean oil and corn oil, at a ratio among the vegetable oil A, the rice oil and the vegetable oil B of 20-35/15-25/40-65 by mass, wherein; the α-linolenic acid content in the constituent fatty acids contained in the oil-and-fat composition is 15 to 30 mass %.

(Vegetable Oil A)

The oil-and-fat composition according to the first embodiment includes at least one vegetable oil selected from flax oil, perilla oil and perilla seed oil. Particularly, it is preferable to use flax oil, and more preferable to use the flax oil having the α-linolenic acid content of not less than 50%, and the trans fatty acid content in the α-linolenic acid of not more than 4 mass %. It is preferable that the trans fatty acid content is decreased as much as possible, since the trans fatty acid is not nutritionally preferable, for example, if it is consumed, an essential fatty acid requirement in vivo is increased.

(Flax Oil)

Flax oil is obtained by pressing flax seeds, and has the α-linolenic acid content of not less than about 50 mass % in the constituent fatty acids, so that it can be preferably used in the embodiment. In the embodiment, particularly, it is preferable to use flax oil having the α-linolenic acid content of not less than 50 mass % in the constituent fatty acids, and the trans fatty acid content in the α-linolenic acid of not more than 4 mass %, for example, it can be manufactured by the following manufacturing condition.

That is to say, the flax oilcan be obtained, which has the α-linolenic acid content of not less than 50 mass % in the constituent fatty acids, and the trans fatty acid content of not more than 4 mass %, if the deodorization process in the manufacturing process of flax oil is carried out in the condition that the deodorization temperature is 220 to 240 degrees C., the deodorization time is 30 to 90 minutes, preferably 50 to 90 minutes and the blowing amount of steam to the oil is 2.0 to 5.5 mass %, preferably 3.0 to 4.5 mass %.

(Rice Oil)

The oil-and-fat composition according to the embodiment includes rice oil. If rice oil is blended together with the vegetable oil A such as flax oil, it can enhance a cooking aptitude, a flavor, and an oxidation stability by compensating the weakness of the vegetable oil A such as flax oil. As the rice oil, distilled rice oil to which alkaki deacidification treatment is not applied can be preferably used, but it is more preferable to use the rice oil to which alkaki deacidification treatment is applied in terms of crystallinity and the like.

(Vegetable Oil B)

The oil-and-fat composition according to the embodiment includes at least one vegetable oil selected from rape seed oil, soybean oil and corn oil. Particularly, it is preferable to use rape seed oil. If the vegetable oil B is blended together with the vegetable oil A such as flax oil, it can enhance a cooking aptitude, a flavor, and an oxidation stability by compensating the weakness of the vegetable oil A such as flax oil.

(Blending Ratio in Oil-and-Fat Composition)

The oil-and-fat composition according to the embodiment includes the vegetable oil A, rice oil and the vegetable oil B at a ratio among the vegetable oil A, the rice oil and the vegetable oil B of 20-35/15-25/40-65 by mass. That is to say, if the other components are not blended, the oil-and-fat composition according to the embodiment includes 20-35 mass % of the vegetable oil A, 15-25 mass % of the rice oil and 40-65 mass % of the vegetable oil B. If the vegetable oil A, the rice oil and the vegetable oil B are blended at the above-mentioned blending ratio, the oil-and-fat composition having a hypertension-ameliorating effect, a good flavor, and good storage stability can be obtained. The oil-and-fat composition according to the embodiment can be prepared by that the respective oil-and-fats stored in the respective tanks are mixed and stirred in a tank for blending.

(α-Linolenic Acid Content)

In the oil-and-fat composition according to the Embodiment, the α-linolenic acid content in the constituent fatty acids contained in the oil-and-fat composition is 15 to 30 mass %. If less than 15 mass %, a hypertension-ameliorating effect can not be highly expected, and if more than 30 mass %, a problem of a flavor is caused.

(Trans Fatty Acid Content in Oil-and-Fat Composition)

The oil-and-fat composition according to the Embodiment has the trans fatty acid content of preferably not more than 3 mass %, and more preferably not more than 2 mass % in the oil-and-fat composition.

(Other Blending Components)

Components to be contained in the oil-and-fat composition according to the embodiment other than the vegetable oil A, the rice oil and the vegetable oil B include sesame oil, sesame salad oil, peanut oil, high-oleic safflower oil, high-linoleic safflower oil, sunflower oil, high-oleic sunflower oil, mid-oleic sunflower oil, high-linoleic sunflower oil, cotton seed oil, grape seed oil, macadamia nuts oil, hazelnut oil, pumpkin seed oil, walnut oil, camellia oil, tea seed oil, borage oil, olive oil, rice bran oil, wheat germ oil, medium chain fatty acid-containing oil, palm oil, palm kernel oil, copra oil, cacao butter, oil-and-fat low saturated by breed improvement, beef fat, lard, chicken fat, milk fat, fish oil, seal oil, alga oil and the like, at least one of them can be used. Further, hydrogenated, fractionated, and ester exchanged oil-and-fat thereof can be also used. Further, emulsifying agent such as monoglycerin fatty acid ester, organic acid monoglycerin fatty acid ester, polyglyceryl fatty acid ester, sucrose fatty acid ester, sorbitan fatty acid ester; ascorbic acid fatty acid ester; lignan; coenzyme Q; phospholipid; orizanol; vitamin E; phytosterol; diglyceride; catechins; and polyphenols can be added. Furthermore, carbohydrate, protein, dietary fiber, vitamin, mineral and the like can be also added.

(Use of Oil-and-Fat Composition and Food or Beverage Containing Oil-and-Fat Composition)

The oil-and-fat composition according to the embodiment can be used for deep-fried food (tempura), stir-frying oil, mold-releasing oil and the like as well as conventional edible oil. A food or beverage containing the oil-and-fat composition according to the embodiment is not particularly limited, and includes food, processed food, health food, beverage, health drink, seasoning, confectionery, pet food, and animals and plants feeding stuff, in particular, gelatin capsule, edible oil, dressing, margarine, prepared margarine, fat spread, cream, ice cream, mayonnaise, bread, cake, doughnut, muffin, scone, fried food, snack food, and diet fluid.

The oil-and-fat composition according to the invention is consumed in an amount of preferably 0.1 g to 20 g, preferably 0.7 g to 14 g, more preferably 1.5 g to 9 g, most preferably 1.8 g to 6 g in adults as an amount of α-linolenic acid per day.

The oil-and-fat composition content in a food or beverage varies according to species of the food or beverage, but the oil-and-fat composition can be contained in the food or beverage in an amount of 0.5% to 100%, and it can be directly used for deep-fried food (tempura), stir-frying oil, mold-releasing oil and the like. The oil-and-fat composition is preferably contained in the food or beverage in an amount of, for example, 0.1 g to 20 g in 100 g of dressing, 7.5 g to 24 g in 100 g of mayonnaise, 6 g to 29.4 g in 100 g of water-in-oil type composition as represented by margarine and fat spread, 0.5 g to 9 g in 100 g of bread, 0.6 g to 7.5 g in 100 g of deep-fried product such as deep-fried food (tempura) and fried food, as an amount of α-linolenic acid.

(Container)

A container of the oil-and-fat composition according to the embodiment includes, for example, can, glass bottle, plastic or paper product, which are heretofore known.

### Advantages of First Embodiment of the Invention

According to the embodiment of the invention, the following advantages are provided.

(1) An edible oil having a high ct-linolenic acid content, a hypertension-ameliorating effect, a good flavor, and good storage stability can be obtained. Here, the hypertension-ameliorating effect means an effect that higher blood pressure than normal blood pressure is lowered so as to be approximated to the normal blood pressure, or to be improved to the normal blood pressure or an optimum blood pressure. The normal blood pressure means that systolic blood pressure is in the range of not less than 120 mmHg and less than 130 mmHg and diastolic blood pressure is in the range of not less than 80 mmHg and less than 85 mmHg, and the optimum blood pressure means that systolic blood pressure is less than 120 mmHg and diastolic blood pressure is less than 80 mmHg.
(2) An edible oil having an excellent aptitude for a cooking (having a good odor at the cooking and a good flavor at the eating) can be obtained, even though it has a high α-linolenic acid content.
(3) A food or beverage containing the edible oil having the above-mentioned effect can be obtained.

[Oil-and-fat composition according to a second Embodiment of the Invention]

An oil-and-fat composition according to a second embodiment of the invention comprises 20-35 mass % of at least one vegetable oil A selected from flax oil, perilla oil and perilla seed oil, and 15-25 mass % of rice oil, wherein the α-linolenic acid content in the constituent fatty acids contained in the oil-and-fat composition is 15 to 30 mass %.

The present embodiment is different from the above-mentioned first embodiment in that it is not indispensable to blend the vegetable oil B, but it is similar to the above-mentioned first embodiment in the other features.

[Oil-and-Fat Composition According to a Third Embodiment of the Invention]

An oil-and-fat composition according to a third embodiment of the invention comprises 30-53 mass % of at least one vegetable oil A selected from flax oil, perilla oil and perilla seed oil and 70-47 mass % of rice oil, wherein the α-linolenic acid content in the constituent fatty acids contained in the oil-and-fat composition is 15 to 30 mass %.

The present embodiment is different from the above-mentioned first embodiment in that the blending of the vegetable oil B is not indispensable, and the ranges of the blending ratio of the vegetable oil A and rice oil are different respectively, but it is similar to the above-mentioned first embodiment in the other features.

### EXAMPLE

Hereinafter, the present invention will be explained by Examples in further detail, but the present invention is not limited by them.

### Examples 1 to 9 and Comparative Examples 1 to 4

Oil-and-fat compositions of Examples 1 to 9 and Comparative Examples 1 to 4 were fabricated at the blending ratios described in Tables 1 to 3 by using flax oil (manufactured by the Nisshin OilliO Group Ltd. by using the method described below), rice oil (manufactured by the Nisshin OilliO Group Ltd. and sold under a trade name of “Nisshin rice salad oil”), rape seed salad oil (manufactured by the Nisshin OilliO Group Ltd. and sold under a trade name of “Nisshin rape seed salad oil (S)”), high-oleic safflower oil (manufactured by the Nisshin OilliO Group Ltd. and sold under a trade name of “Nisshin safflower oil”), and corn oil (manufactured by the Nisshin OilliO Group Ltd. and sold under a trade name of “Nisshin corn oil”).

[Manufacturing Method of Flax Oil]

After flax seeds (produced in Canada) were heated to 80 degrees C., an expression treatment was applied so as to obtain flax crude oil, and 3 mass % of water to the crude oil was added at 70 degrees C. while being stirred and maintained for 20 minutes, and then, a centrifugal separation was carried out at 3000×G for 20 minutes so as to precipitate and eliminate water dispersible phospholipid, and clear upper portion obtained was dried by being heated to 100 degrees C. under reduced pressure by using a vacuum pump, so as to obtain degumming oil. Next, 0.1 mass % of phosphoric acid having a concentration of 75 volume % to the degumming oil was added to the degumming oil, after being maintained for 15 minutes while continuously being stirred at 70 degrees C., acid value was measured, next, an aqueous solution of sodium hydroxide having Baume degree of 16 was added in a 50% excessive amount than an amount needed for carrying out saponification of free fatty acid, after being maintained for 15 minutes while continuously being stirred at 70 degrees C., a centrifugal separation was carried out at 3000×G for 20 minutes, and after a mixture of non-water dispersible phospholipid and fatty acid soap was precipitated and eliminated as oil cake, water washing was repeated until water washing liquid is neutralized, so as to carry out deacidification treatment. After that, drying was carried out by being heated to 100 degrees C. under reduced pressure by using a vacuum pump. Subsequently, after 1 mass % of activated white earth (manufactured by Mizusawa Chemicals, Ltd. to the dried oil was added to the dried oil and maintained at 110 degrees C. for 20 minutes under reduced pressure by using a vacuum pump, the white earth was separated by filtration at 85 degrees C., so as to carry out decolorization treatment. Further, steam blowing deodorization treatment (total amount of steam blowing: 5 mass % to oil) was carried out at 230 degrees C. for 60 minutes, so as to fabricate purified flax oil having the α-linolenic acid content of 56.3 mass % and the trans fatty acid content of 2.5 mass %.

With regard to each of the obtained oil-and-fat compositions of Examples 1 to 9 and Comparative Examples 1 to 4, evaluations of flavor and cooked odor were carried out according to the evaluation test method described below. Evaluation results are shown in Tables 1 to 3.

[Evaluation of Flavor]

The evaluation of flavor was carried out by that a panel of seven experts put almost 1 to 2 ml of the oil-and-fat composition in a state of normal temperature into their mouth, so as to evaluate according to the criterion for evaluation (on a scale of 1 to 5) described below, and the average value was determined as the evaluation value.

### (Criterion for Evaluation)

Scale of 5: very good
Scale of 4: goodish
Scale of 3: common
Scale of 2: baddish
Scale of 1: very bad

[Evaluation of Heated Odor (Cooked Odor)]

The evaluation of heated odor (cooked odor) was carried out by that 50 g of the oil-and-fat composition were fed in a beaker of 100 ml in volume and heated to 120 degrees C., and a panel of seven experts evaluated according to the criterion for evaluation (on a scale of 1 to 5) described below, and the average value was determined as the evaluation value.

### (Criterion for Evaluation)

Scale of 5: very good
Scale of 4: goodish
Scale of 3: common
Scale of 2: baddish
Scale of 1: very bad

Further, with regard to each of the obtained oil-and-fat compositions, storage test was carried out according to the storage test method described below. Evaluation results are shown in Tables 1 to 3.

[Evaluation of Storage Stability]

The evaluation of storage stability was carried out by that the oil-and-fat composition was fed in a PET container of 600 g in volume, and stored at the storing temperature of 40 degrees C. for 12 weeks and stored at the storing temperature of 20 degrees C. and under the illuminance of 1000 lux for 12 weeks, and then flavor and cooked odor were evaluated as well as the above description.

[Table 1]

[Table 2]

[Table 3]

Further, with regard to each of the obtained oil-and-fat compositions of Examples 1 to 9 and Comparative Examples 1 to 4, cooking test was carried out according to the cooking test method described below. Evaluation results are shown in Tables 4 to 6.

[Evaluation of Cooking]

Each of the food items described in Tables 4 to 6 was cooked in the usual manner, odor at the cooking and flavor at the eating were evaluated. The evaluation was carried out by a panel of seven experts according to the criterion for evaluation (on a scale of 1 to 5) described below, and the average value was determined as the evaluation value.

### (Criterion for Evaluation)

Scale of 5: very good
Scale of 4: goodish
Scale of 3: common
Scale of 2: baddish
Scale of 1: very bad

[Table 4]

[Table 5]

[Table 6]

As is clear from Tables 1 to 6, the oil-and-fat compositions of Examples 1 to 9 according to the invention have a good flavor before and after the storage and do not have an unpleasant odor at the heating, and further, have a good odor at the cooking and a good flavor at the eating.

On the other hand, it is known that the oil-and-fat compositions of the Comparative Examples 1 to 4 of which blending component, blending ratio or α-linolenic acid content is out of the range defined in the present invention are inferior in any of flavor and cooked odor before and after the storage, and odor at the cooking and flavor at the eating to those of Examples 1 to 9.

### Example 10, Comparative Example 5, Reference Example 1

Next, the hypertension-ameliorating effect verification test described below was carried out by using the above-mentioned oil-and-fat composition of Example 2 (Example 10), salad oil moderately containing α-linolenic acid (Comparative Example 5) and high-oleic safflower oil (manufactured by the Nisshin OilliO Group Ltd. and sold under a trade name of “Nisshin safflower oil”) (Reference Example 1) as test oils. The high-oleic safflower oil contains very little α-linolenic acid so that it is used as a reference oil. As the salad oil moderately containing α-linolenic acid, a mixture of salad oil (manufactured by the Nisshin OilliO Group Ltd. and sold under a trade name of “Nisshin soybean salad oil (S)”) and salad oil (manufactured by the Nisshin OilliO Group Ltd. and sold under a trade name of “Nisshin rape seed salad oil (S)”) having a mixing ratio of 1:1 was used for the test.

[Hypertension-Ameliorating Effect Verification Test by a Forced Oral Administration to Rats]

As a test animal, 5-week-old and male SHR/Izm being a hypertension pathogenesis model rat was used, and after a preliminary breeding of 6 days, 1 ml (0.92 g) of the test oil was forcibly and orally administered by using a stomach sound. After 4 hours from the forced oral administration, a cuff was wrapped around the tail portion of the rat, and the blood pressure was measured by using a non-invasive blood pressure monitor (manufactured by Muromachi Kikai Co., Ltd. and sold under a model number of “MK-2000”). As the feeding stuff during the preliminary breeding and test, solid feed (manufactured by PMI Nutrition International and sold under a trade name of “PicoLab Rodent Diet 20”) was freely consumed and drinkable water was also freely consumed. The blood pressure of the rat by which each of the test oils was consumed after 4 hours from the forced oral administration are shown in Table 7. Further, the fatty acid composition of each of the test oils is shown in Table 8.

[Table 7]

The blood pressure after 4 hours from the forced oral administration were shown by the average value±standard deviation. Between groups having different alphabets (a, b), statistically significant differences (P<0.05) were seen. Statistical processing was carried out by Bonferroni multiple comparison calibration.

As is clear from Table 7, it was verified that the oil-and-fat composition of Example 2 (Example 10) has a hypertension-ameliorating effect on rats, since the blood pressure of rat in case of Example 10 was significantly decreased in comparison with the blood pressures in cased of the salad oil (Comparative Example 5) and the high-oleic safflower oil (Reference Example 1).

On the other hand, the salad oil moderately containing α-linolenic acid (Comparative Example 5) has a tendency that the blood pressure is decreased in comparison with the high-oleic safflower oil (Reference Example 1), but no significant differences are recognized.

[Table 8]

TABLE 1

| | | Example 1 | Example 2 | Example 3 | Example 4 |
|---|---|---|---|---|---|
| Blending oil and | | Flax/Rice/ | Flax/Rice/ | Flax/Rice/ | Flax/Rice/ |
| Blending ratio | | Rape- | Rape- | Rape- | Rape- |
| | | seed = | seed = | seed = | seed = |
| | | 20/20/60 | 25/20/55 | 30/20/50 | 35/20/45 |
| α-linolenic acid | | 17.3% | 19.6% | 21.8% | 24.1% |
| content | | | | | |
| Before | Flavor | 4.6 | 4.7 | 4.6 | 4.6 |
| storage | Cooked | 4.6 | 4.4 | 4.6 | 4.6 |
| | odor | | | | |
| After | Flavor | 4.1 | 4.0 | 4.1 | 4.0 |
| storage 1 | Cooked | 4.0 | 4.1 | 4.0 | 4.1 |
| | odor | | | | |
| After | Flavor | 4.1 | 4.1 | 4.1 | 4.0 |
| storage 2 | Cooked | 4.0 | 4.0 | 4.1 | 4.1 |
| | odor | | | | |

(Notes)
After storage 1: after stored at 40 degrees C. for 12 weeks
After storage 2: after stored at 20 degrees C. and under 1000 lux for 12 weeks

TABLE 2

| | | Example 5 | Example 6 | Example 7 | Example 8 | Example 9 |
|---|---|---|---|---|---|---|
| Blending oil and | | Flax/Rice/ | Flax/Rice/ | Flax/Rice/ | Flax/Rice = | Flax/Rice = |
| Blending ratio | | Rapeseed = | Rapeseed = | Rapeseed = | 30/70 | 50/50 |
| | | 35/15/50 | 25/15/60 | 25/25/50 | | |
| α-linolenic acid | | 24.6% | 20.1% | 19.1% | 16.8% | 27.8% |
| content | | | | | | |
| Before | Flavor | 4.4 | 4.7 | 4.7 | 4.7 | 4.7 |
| storage | Cooked | 4.4 | 4.3 | 4.9 | 4.7 | 4.6 |
| | odor | | | | | |
| After | Flavor | 4.0 | 4.3 | 4.1 | 4.1 | 4.3 |
| storage 1 | Cooked | 4.0 | 4.0 | 4.6 | 4.3 | 4.1 |
| | odor | | | | | |
| After | Flavor | 4.0 | 4.0 | 4.3 | 4.3 | 4.1 |
| storage 2 | Cooked | 4.0 | 4.0 | 4.0 | 4.1 | 4.1 |
| | odor | | | | | |

(Notes)
After storage 1: after stored at 40 degrees C. for 12 weeks
After storage 2: after stored at 20 degrees C. and under 1000 lux for 12 weeks

TABLE 3

| | | Com. Ex. 1 | Com. Ex. 2 | Com. Ex. 3 | Com. Ex. 4 | Com. Ex. 5 | Com. Ex. 6 |
|---|---|---|---|---|---|---|---|
| Blending oil and | | Flax/Rice/ | Flax/ | Flax/High-oleic | Flax/Corn/ | Flax/Corn/ | Flax/Rice/ |
| Blending ratio | | Rapeseed = | Rapeseed = | safflower oil = | Rapeseed = | Rapeseed = | Rapeseed = |
| | | 50/10/40 | 50/50 | 60/40 | 20/60/20 | 30/30/40 | 40/20/40 |
| α-linolenic acid | | 31.8% | 32.5% | 33.1% | 13.2% | 20.1% | 26.4 |
| content | | | | | | | |
| Before | Flavor | 3.6 | 3.4 | 3.4 | 4.0 | 3.7 | 3.7 |
| storage | Cooked | 3.7 | 3.3 | 3.4 | 3.9 | 3.9 | 3.7 |
| | odor | | | | | | |
| After | Flavor | 2.6 | 2.4 | 2.9 | 3.3 | 3.1 | 3.1 |
| storage 1 | Cooked | 2.7 | 2.1 | 2.7 | 3.4 | 3.1 | 3.3 |
| | odor | | | | | | |
| After | Flavor | 2.1 | 2.0 | 2.4 | 3.1 | 3.1 | 3.1 |
| storage 2 | | | | | | | |

(Notes)
Com. Ex.: Comparative Example
After storage 1: after stored at 40 degrees C. for 12 weeks
After storage 2: after stored at 20 degrees C. and under 1000 lux for 12 weeks

TABLE 4

| | Example 1 | Example 2 | Example 3 | Example 4 |
|---|---|---|---|---|
| Blending oil and | Flax/Rice/ | Flax/Rice/ | Flax/Rice/ | Flax/Rice/ |
| Blending ratio | Rapeseed = | Rapeseed = | Rapeseed = | Rapeseed = |
| | 20/20/60 | 25/20/55 | 30/20/50 | 35/20/45 |
| Deep-fried | 4.7 | 4.7 | 4.9 | 4.4 |
| sweet potato | | | | |
| Deep-fried | 4.7 | 4.6 | 4.6 | 4.6 |
| shrimp | | | | |
| Croquette | 4.6 | 4.6 | 4.9 | 4.6 |
| Fried chicken | 4.6 | 4.6 | 4.6 | 4.6 |
| Scramble eggs | 4.7 | 4.7 | 4.4 | 4.6 |
| Stir-fried | 4.6 | 4.7 | 4.6 | 4.4 |
| vegetables | | | | |
| Dressing | 4.6 | 4.6 | 4.4 | 4.6 |

TABLE 5

| | Example 5 | Example 6 | Example 7 | Example 8 | Example 9 |
|---|---|---|---|---|---|
| Blending oil | Flax/Rice/ | Flax/Rice/ | Flax/Rice/ | Flax/ | Flax/ |
| and Blending | Rape- | Rape- | Rape- | Rice = | Rice = |
| ratio | seed = | seed = | seed = | 30/70 | 50/50 |
| | 35/15/50 | 25/15/60 | 25/25/50 | | |
| Deep-fried | 4.2 | 4.3 | 4.7 | 4.9 | 4.4 |
| sweet potato | | | | | |
| Deep-fried | 4.4 | 4.6 | 4.7 | 4.6 | 4.7 |
| shrimp | | | | | |
| Croquette | 4.4 | 4.6 | 4.4 | 4.7 | 4.6 |
| Fried chicken | 4.2 | 4.6 | 4.6 | 4.6 | 4.6 |
| Scramble | 4.4 | 4.4 | 4.6 | 4.6 | 4.6 |
| eggs | | | | | |
| Stir-fried | 4.4 | 4.4 | 4.6 | 4.7 | 4.6 |
| vegetables | | | | | |
| Dressing | 4.4 | 4.6 | 4.7 | 4.6 | 4.6 |

TABLE 6

| | Com. Ex. 1 | Com. Ex. 2 | Com. Ex. 3 | Com. Ex. 4 | Com. Ex. 5 | Com. Ex. 6 |
|---|---|---|---|---|---|---|
| Blending oil and | Flax/Rice/ | Flax/ | Flax/High-oleic | Flax/Corn/ | Flax/Corn/ | Flax/Rice/ |
| Blending ratio | Rapeseed = | Rapeseed = | safflower oil = | Rapeseed = | Rapeseed = | Rapeseed = |
| | 50/10/40 | 50/50 | 60/40 | 20/60/20 | 30/30/40 | 40/20/40 |
| Deep-fried sweet | 3.4 | 3.1 | 3.3 | 3.6 | 3.3 | 3.4 |
| potato | | | | | | |
| Deep-fried | 3.6 | 3.0 | 3.3 | 3.4 | 3.4 | 3.4 |
| shrimp | | | | | | |
| Croquette | 3.1 | 2.8 | 3.1 | 3.7 | 3.1 | 3.3 |
| Fried chicken | 3.1 | 3.1 | 3.0 | 3.4 | 3.0 | 3.1 |
| Scramble eggs | 3.1 | 3.1 | 3.1 | 3.7 | 3.4 | 3.6 |
| Stir-fried | 3.0 | 3.3 | 3.1 | 3.4 | 3.4 | 3.4 |
| vegetables | | | | | | |
| Dressing | 3.4 | 3.3 | 3.4 | 3.6 | 3.6 | 3.4 |

(Notes)
Com. Ex.: Comparative Example

TABLE 7

| | | Blood pressure after 4 |
|---|---|---|
| | | hours from forced oral |
| | Test oil | administration (mmHg) |
| Example 10 | Oil-and-fat composition of | 142.1 ± 9.6b |
| | Example 2 | |
| Com. Ex. 5 | Salad oil | 159.4 ± 13.4a |
| Ref. Ex. 1 | High-oleic safflower oil | 167.2 ± 8.1a |

(Notes)
Com. Ex.: Comparative Example
Ref. Ex.: Reference Example

TABLE 8
Fatty acid composition of test oil (%)

| | Example 10 | Com. Ex. 5 | Ref. Ex. 1 |
|---|---|---|---|
| 16:0 | 6.9 | 7.5 | 4.9 |
| 18:0 | 2.1 | 2.9 | 2.0 |
| 18:1n-9 | 46.0 | 41.3 | 77.5 |
| 18:2n-6 | 22.3 | 36.9 | 13.7 |
| 18:3n-3 | 19.9 | 8.7 | 0.6 |
| 20:0 | 0.5 | 0.5 | 0 |
| 20:1n-9 | 0.8 | 0.7 | 0.4 |
| 22:0 | 0.2 | 0.4 | 0.3 |
| 24:0 | 0.1 | 0.2 | 0.2 |
| Others | 1.2 | 0.9 | 0.4 |

(Notes)
Com. Ex.: Comparative Example
Ref. Ex.: Reference Example

1. An oil-and-fat composition, comprising:
at least one vegetable oil A selected from flax oil, perilla oil and perilla seed oil;
rice oil; and
at least one vegetable oil B selected from rape seed oil, soybean oil and corn oil, at a ratio among the vegetable oil A, the rice oil and the vegetable oil B of 20-35/15-25/40-65 by mass, wherein:
the α-linolenic acid content in the constituent fatty acids contained in the oil-and-fat composition is 15 to 30 mass %.

2. An oil-and-fat composition, comprising:
20-35 mass % of at least one vegetable oil A selected from flax oil, perilla oil and perilla seed oil; and
15-25 mass % of rice oil, wherein:
the α-linolenic acid content in the constituent fatty acids contained in the oil-and-fat composition is 15 to 30 mass %.

3. An oil-and-fat composition, comprising:
30-53 mass % of at least one vegetable oil A selected from flax oil, perilla oil and perilla seed oil; and
70-47 mass % of rice oil, wherein:
the α-linolenic acid content in the constituent fatty acids contained in the oil-and-fat composition is 15 to 30 mass %.

4. The oil-and-fat composition, according to claim wherein:
the vegetable oil A is flax oil.

5. The oil-and-fat composition, according to claim 1, wherein:
the vegetable oil B is rape seed oil.

6. The oil-and-fat composition, according to claim 4 wherein:
the trans fatty acid content in the oil-and-fat composition is not more than 3 mass %.

7. A food or beverage containing the oil-and-fat composition according to claim 6.

8. The oil-and-fat composition, according to claim 2, wherein:
the vegetable oil A is flax oil.

9. The oil-and-fat composition, according to claim 8, wherein:
the trans fatty acid content in the oil-and-fat composition is not more than 3 mass %.

10. A food or beverage containing the oil-and-fat composition according to claim 9.

11. The oil-and-fat composition, according to claim 3, wherein:
the vegetable oil A is flax oil.

12. The oil-and-fat composition, according to claim 11, wherein:
the trans fatty acid content in the oil-and-fat composition is not more than 3 mass %.

13. A food or beverage containing the oil-and-fat composition according to claim 12.
